(19) United States
(12) Patent Application Publication
(10) Pub. No.: US 2012/0289848 A1
(43) Pub. Date: Nov. 15, 2012

(54) METHOD AND SYSTEM FOR DISCRIMINATING HEART SOUND AND CARDIOPATHY

(75) Inventors: Arvin Huang-Te Li, Chiayi City (TW); Yio-Wha Shau, Taipei City (TW)

(73) Assignee: INDUSTRIAL TECHNOLOGY RESEARCH INSTITUTE, HSINCHU (TW)

(21) Appl. No.: 13/288,547

(22) Filed: Nov. 3, 2011

(30) Foreign Application Priority Data
May 10, 2011 (TW) 100116406

(51) Int. Cl. A61B 7/00

(52) U.S. Cl. 600/528

(57) ABSTRACT

A method for discriminating heart sound is provided. The method comprises the following steps. A heart-sound signal is provided. A specific function calculation is performed on the heart-sound signal to generate a first calculation signal and suppress the noise of the heart-sound signal. The filtering signal is transformed to generate data for an image plots. The image plot corresponding to the data generated in the previous step is generated and compared with data of heart-sound plots and the comparison result is used for discriminating the heart sound.

This application claims the benefit of Taiwan application Serial No. 100116406, filed May 10, 2011, the subject matter of which is incorporated herein by reference.

### BACKGROUND

1. Technical Field

The disclosure relates in general to a method and system for discriminating heart sound and cardiopathy.

2. Description of the Related Art

Conventional cardiovascular disease can be diagnosed by detecting the state of patient's heart sound with an electronic stethoscope. However, when detecting the patient's heart-sound, background noises, such as the patient's conversation with hospital staff and the sound of friction or collision generated when moving furniture, are recorded at the same time. Thus, before analyzing the detected heart-sound signal, the detected heart sound signal and the noise component must be separated first to assure the correctness of the analysis result.

Various conventional filters and algorithms, such as the short time Fourier transform (STFT) algorithm, the Hilbert-Huang transform (HHT) algorithm and the wavelet transform (WT) algorithm, are used to separate the heart-sound signal from the noise. However, effective separation still cannot be achieved. Particularly, when the conventional algorithms are used, some minor heart-sound signals will be covered by the noise, and the doctor cannot clearly and correctly diagnose cardiopathy diseases according to the obtained phonocardiogram (PCG). Therefore, how to effectively separate the to-be-detected heart-sound signal from the noise has become an imminent task in the diagnosis of cardiopathy.

### SUMMARY

The disclosure is directed to a method and system for discriminating heart sound and cardiopathy.

In some embodiments of the present disclosure, a method for discriminating heart sound is provided. The method comprises the following steps. A heart-sound signal is provided. A specific function calculation is performed on the heart-sound signal to generate a first calculation signal and suppress the noise of the heart-sound signal. The filtering signal is transformed to generate data for an image plots. The image plot corresponding to the data generated in the previous step is generated and compared with data of heart-sound plots and the comparison result is used for discriminating the heart sound.

In other embodiments of the present disclosure, a method for discriminating cardiopathy is provided. The method comprises the following steps: A heart-sound signal is received. A specific function calculation is performed on the heart-sound signal to generate a first calculation signal. The first calculation signal is filtered to generate a filtering signal. The filtering signal is transformed to generate data for an image plot. The image plot corresponding to the data generated in the previous step is generated and compared with data of cardiopathy heart-sound plots, and the comparison result is used for cardiopathy discrimination.

In some embodiments of the present disclosure, a system for discriminating heart sound and cardiopathy comprising a signal receiving unit and a signal processing unit is provided. The signal receiving unit receives a heart-sound signal. The signal processing unit comprises a first calculation unit, a filter unit and a second calculation unit. The first calculation unit is coupled to the signal receiving unit for performing a specific function calculation on the heart-sound signal to generate a first calculation signal. The filter unit is coupled to the first calculation unit for filtering the first calculation signal to generate a filtering signal. The second calculation unit is coupled to the filter unit for performing a transformation calculation on the filtering signal to generate data for to an image plot.

The above and other aspects of the disclosure will become better understood with regard to the following detailed description of the non-limiting embodiment(s). The following description is made with reference to the accompanying drawings.

### BRIEF DESCRIPTION OF THE DRAWINGS

FIG. 1 is a flowchart of a method for discriminating heart sound according to an embodiment of the invention;

FIGS. 2A˜2D are respectively time-frequency plots obtained by performing a calculation on a normal first sound of the heart-sound signal according to the method for discriminating heart sound in the embodiment of the invention and the conventional HHT, STFT and WT algorithms;

FIGS. 3A˜3D are respectively time-frequency plots obtained by performing a calculation on a widely split second sound of the heart-sound signal according to the method for discriminating heart sound in the embodiment of the invention and the conventional HHT, STFT and WT algorithms;

FIGS. 4A˜4D are respectively time-frequency plots obtained by performing a calculation on a midsystolic murmur of the heart-sound signal according to the method for discriminating heart sound in the embodiment of the invention and the conventional HHT, STFT and WT algorithms;

FIG. 5 is a flowchart of a method for discriminating cardiopathy according to an embodiment of the invention;

FIGS. 6A˜6H are an example of a database of cardiopathy heart-sound plots according to an embodiment of the invention; and

FIG. 7 is a block diagram of a system for discriminating heart sound and cardiopathy according to an embodiment of the invention.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The disclosure relates to a method and system for discriminating heart sound and cardiopathy. According to the embodiments of the disclosed method and system, a specific function is performed on a heart-sound signal to decrease the noise, and a filter is used to filter the noise mixed in the heart-sound signal. Then, the HHT algorithm is performed and then the STFT algorithm is selectively performed to obtain a required time-frequency plot, so that the to-be-detected heart-sound signal is separated from the noise to help discriminating the heart-sound signal. Furthermore, the heart-sound signal is compared with the database of heart-sound plots and/or cardiopathy heart-sound plots, and the comparison result enables the doctor to make prompt and correct analysis and diagnosis of diseases.

In some embodiments of the present disclosure, a method for discriminating heart sound is provided. The method comprises the following steps. A heart-sound signal including a plurality of heart-sound frequencies is provided. A specific function calculation is performed on the heart-sound signal to generate a first calculation signal and suppress the noise of the heart-sound signal. The filtering signal is transformed to generate data corresponding to an image plots. The image plot corresponding to the data generated in the previous step is generated and compared with data of heart-sound plots and the comparison result is used to discriminate the heart sound.

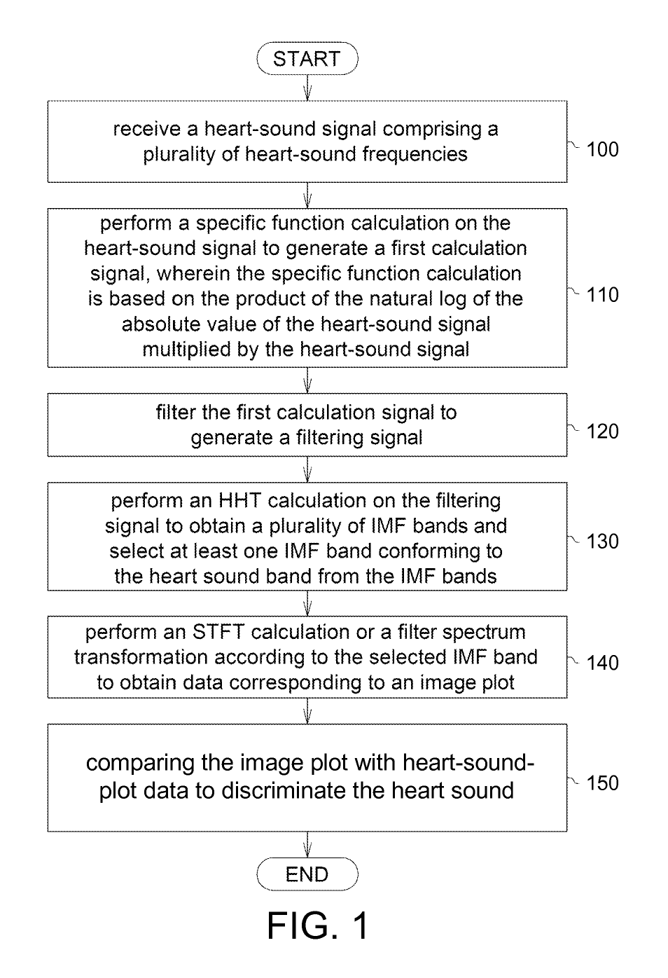

Referring to FIG. 1, a flowchart of a method for discriminating heart sound according to an embodiment of the invention is shown. Firstly, the method begins at step 100, a heart-sound signal A is received by a signal receiving unit (such as an electronic stethoscope), wherein the heart-sound signal A comprises a plurality of heart-sound frequencies. Next, the method proceeds to step 110, a specific function calculation is performed on the heart-sound signal A by a first calculation unit to generate a first calculation signal X, wherein the specific function calculation is based on the product of the natural log of the absolute value of the heart-sound signal A multiplied by the heart-sound signal A, such as expressed in formula (1) with c being any value or function value.

X=cAln|A′| formula (1)

Wherein c can be any value or function value, A′=A if A≠0, and A′=R if A=0, R≧1, R is a real number. In the first calculation signal X processed by the aforementioned specific function calculation, the noise is reduced and the part of the real heart-sound to be detected is enhanced.

Then, the method proceeds to step 120, the first calculation signal X is filtered by a filter unit (such as a median filter) to generate a filtering signal Y as indicated in formula (2).

Y[p,q]=median{X[i,j],(i,j)εW} formula (2)

Wherein i, j, p, q denote the size of the matrix, and W denotes the range of the matrix.

In the filtering signal Y obtained by smoothing the first calculation signal X with a median filter, the noise of the heart sound signal is reduced to a minimum or is completely eliminated (see References [1]-[3]).

Then, the method proceeds to step 130, an HHT calculation is performed on the filtering signal Y by the second calculation unit 126 to obtain a number of IMF bands IMF1, IMF2, IMF3 . . . through mode decomposition, at least one IMF band conforming to the heart sound band normally being the second digital IMF2 is selected from the IMF bands (see References [4]-[6]).

Then, the method proceeds to step 140, the STFT calculation as indicated in formula (3) (see References [7]-[8]) or the filter spectrum transformation is performed by the second calculation unit 126 according to the selected IMF band to obtain the data Z corresponding to an image plot (that is, time frequency plot).

$$STFT \{ z ( t ) \} ≡ Z ( τ , w ) = {\int }_{- ∞}^{∞} z ( t ) w ( t - τ ) exp ( - j ω t ) d t \qquad formula ( 3 )$$

Wherein, z (t) denotes an IMF2 value, w denotes a window function, and T denotes time.

The data Z obtained in the previous step is converted into a plot, wherein the horizontal axis denotes a time axis, the vertical axis denotes a frequency band, and the color darkness denotes segment intensity, and a time-frequency plot required for discriminating the heart sound is thus completed.

Lastly, the method proceeds to step 150, the image plot (that is, a time-frequency plot) is correspondingly generated according to the data Z generated in step 140, the image plot is compared with heart-sound-plot data, and the comparison result can be used to discriminate heart sound. The aforementioned data Z corresponding to the image plot could be output/transmitted to a display. The transmission way could be wireless transmission (such as Bluetooth transmission, WiFy) or wired transmission interface (such as a USB interface or an RS232 or a 1394 transmission line) to display the required image plot.

According to the method for discriminating heart sound of the present embodiment of the invention disclosed above, the aforementioned specific function calculation is performed on the heart-sound signal, which is further filtered so that the noise is reduced to a minimum or completely eliminated, and the discrimination of the real heart-sound signal component is improved. The above points are verified in a number of clinical examples below.

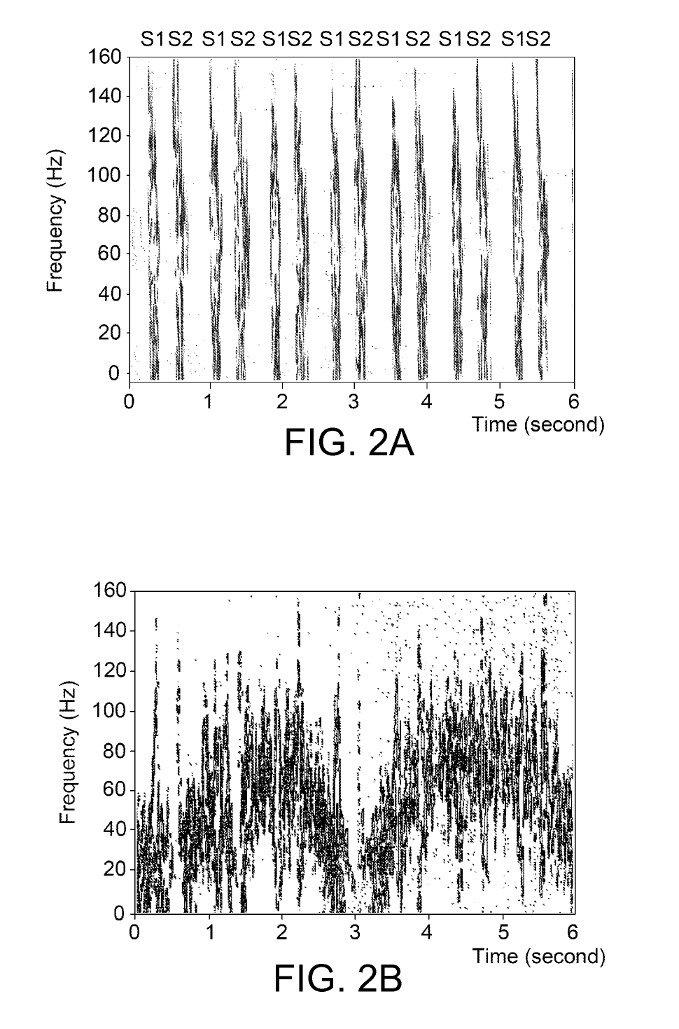

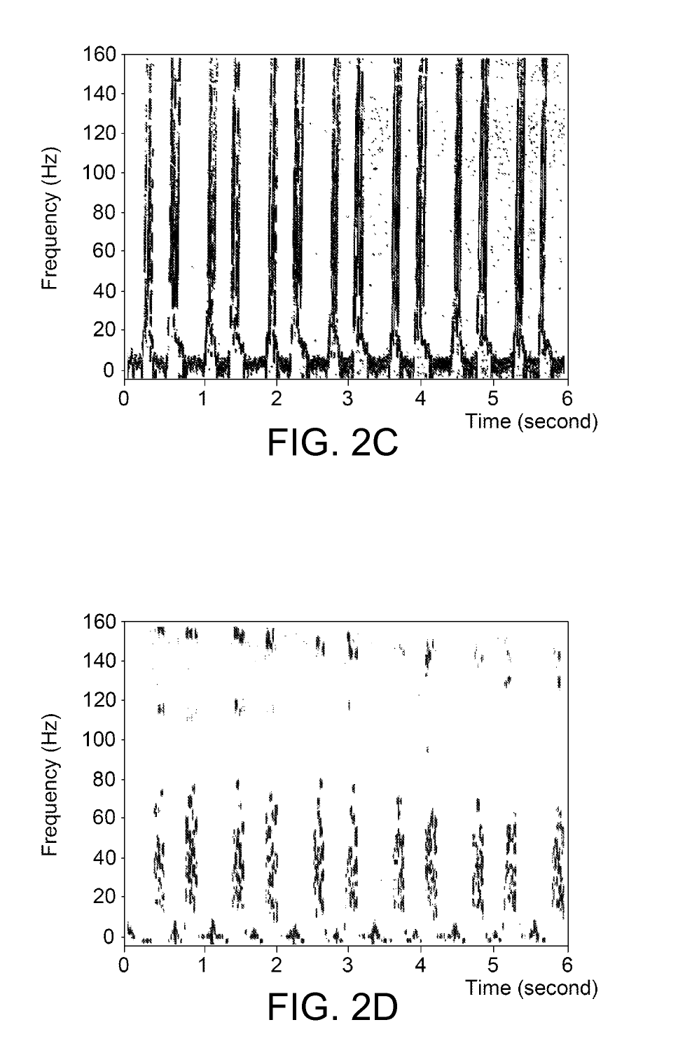

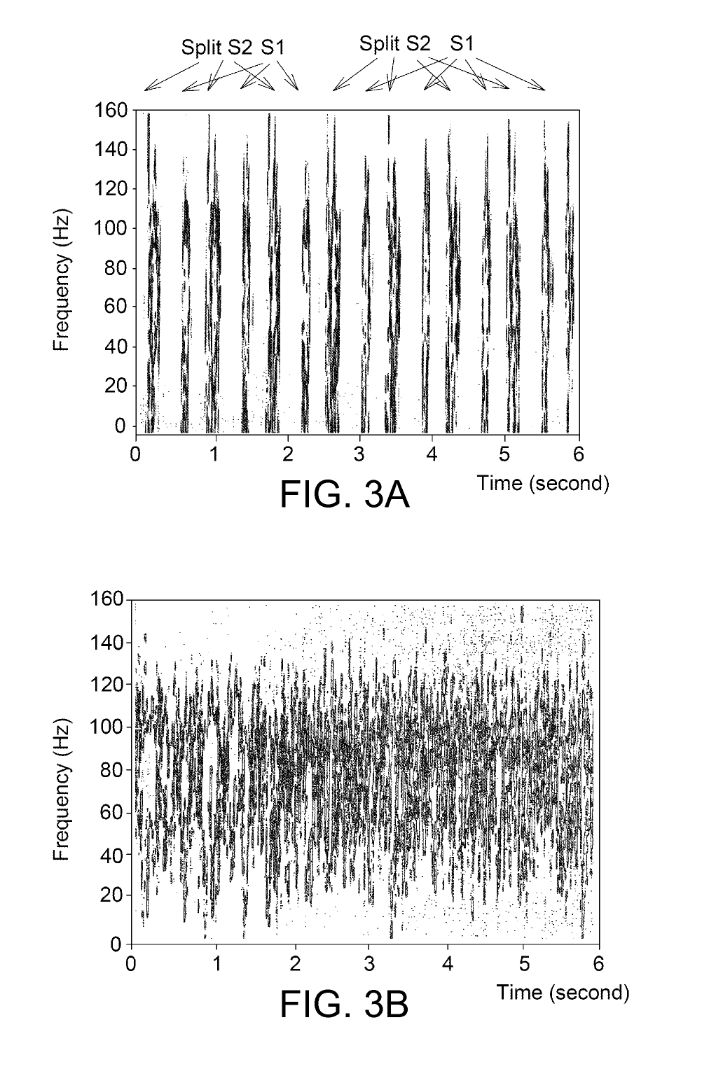

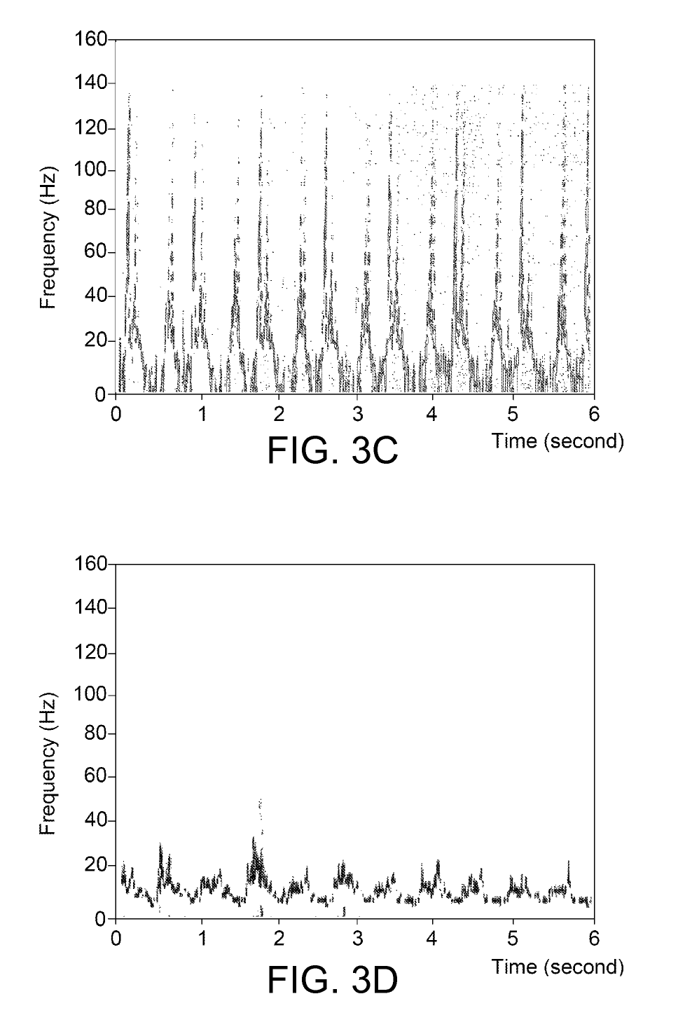

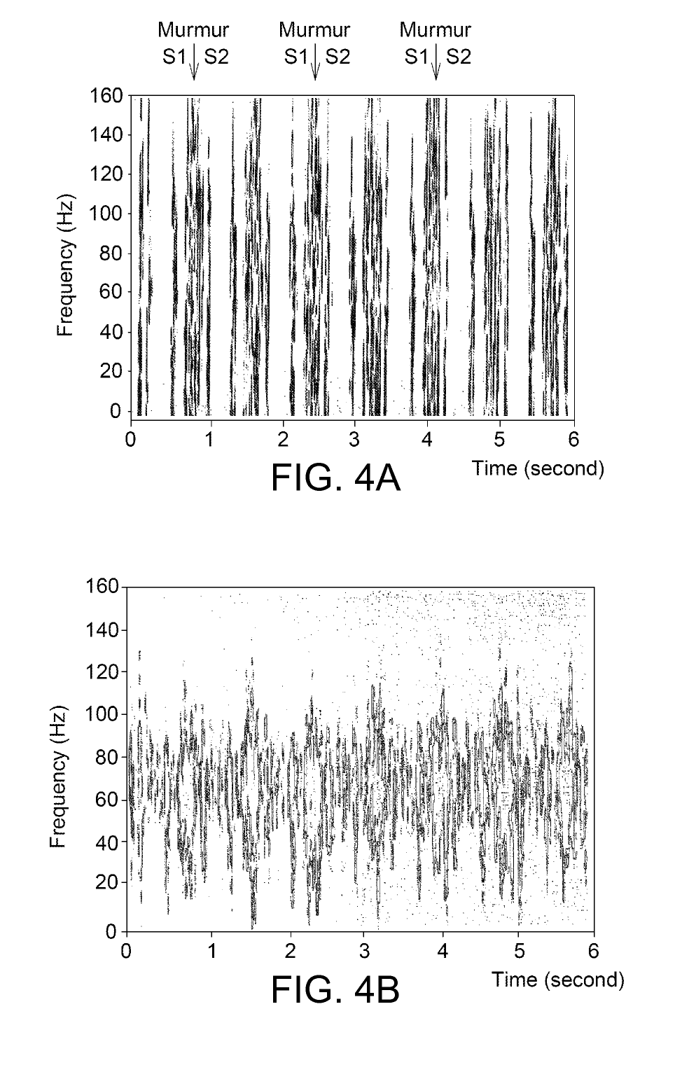

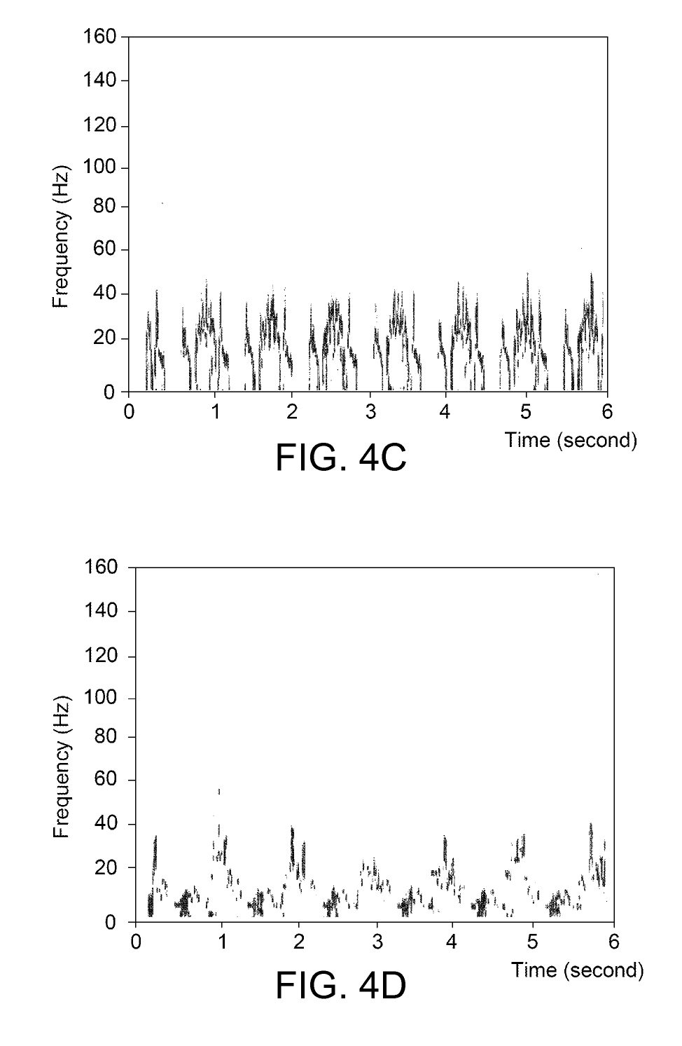

FIGS. 2A˜2D respectively are the time-frequency plots obtained by performing a calculation on a normal first sound S1 of the heart-sound signal according to the method for discriminating heart sound in the embodiment of the invention and the conventional HHT, STFT and WT algorithms. The normal first sound S1 mainly refers to the closing snap of mitral and tricuspid. FIGS. 3A˜3D respectively are the time-frequency plots obtained by performing a calculation on a widely split second sound S2 of the heart-sound signal according to the method for discriminating heart sound in the embodiment of the invention and the conventional HHT, STFT and WT algorithms, wherein the widely split second sound S2 is currently regarded as relating to right bundle-branch block or pulmonary stenosis. FIGS. 4A˜4D respectively are the time-frequency plots obtained by performing a calculation on a midsystolic murmur of the heart-sound signal according to the method for discriminating heart sound in the preferred embodiment of the invention and the conventional HHT, STFT and WT algorithms. The midsystolic murmur indicates severe aortic stenosis which arises when aortic valves are thickened and stuck together.

As indicated in FIGS. 2A˜2D, FIGS. 3A˜3D and FIGS. 4A˜4D, the doctor can discriminate the to-be-detected characteristic signal more easily from the time-frequency plots (FIG. 2A, FIG. 3A and FIG. 4A) obtained according to the method for discriminating heart sound of the present embodiment of the invention than from the time-frequency plots (FIGS. 2B˜2D, FIGS. 3B˜3D and FIGS. 4B˜4D) obtained by the conventional HHT, STFT and WT algorithms.

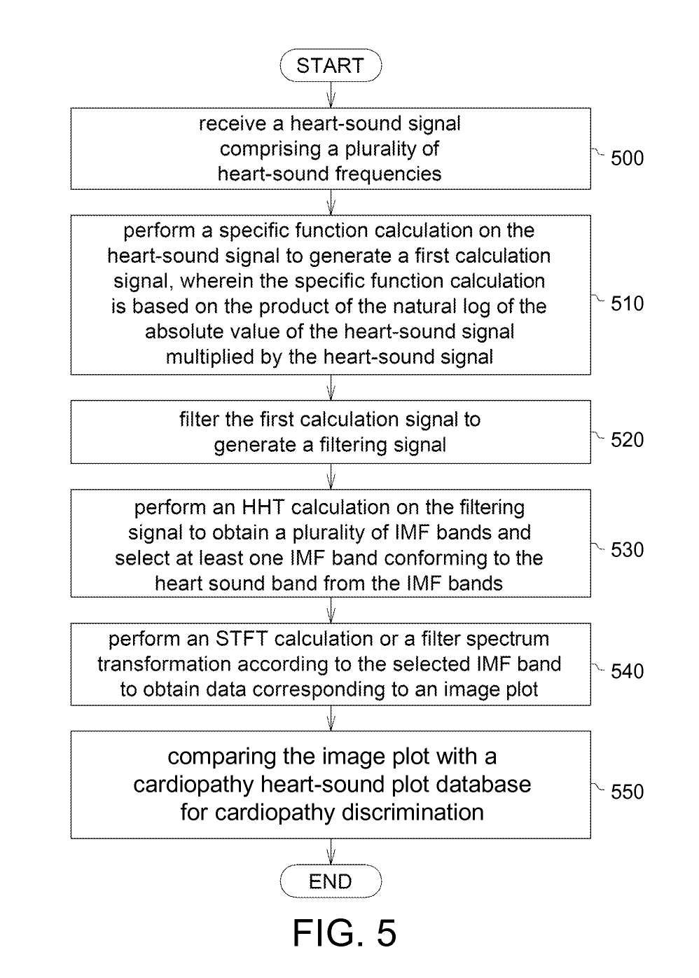

Referring to FIG. 5, a flowchart of a method for discriminating cardiopathy according to an embodiment of the invention is shown. Firstly, the method begins at step 500, a heart-sound signal A is received by a signal receiving unit (such as an electronic stethoscope), wherein the heart-sound signal A comprises a number of heart-sound frequencies. Next, the method proceeds to step 510, a specific function calculation is performed on the heart-sound signal A by a first calculation unit to generate a first calculation signal, wherein the specific function calculation is based on the product of the natural log of the absolute value of the heart-sound signal A multiplied by the heart-sound signal A, such as indicated in formula (1) with c being any value or function value, A′=A if A≠0, and A′=R if A=0, R≧1, R is a real number. In the first calculation signal X processed by the aforementioned specific function calculation, the noise is reduced and the part of the real heart-sound to be detected is enhanced.

Then, the method proceeds to step 520, the first calculation signal X is filtered by a filter unit (such as a median filter) to generate a filtering signal Y as indicated in formula (2). In the filtering signal Y obtained by smoothing the first calculation signal X with a median filter, the noise of the heart sound signal is reduced to a minimum or completely eliminated.

Then, the method proceeds to step 530, an HHT calculation is performed on the filtering signal Y by a second calculation unit to obtain a plurality of IMF bands through mode decomposition, at least one IMF band conforming to the heart sound signal band is selected from the IMF bands.

Then, the method proceeds to step 540, an STFT calculation as indicated in formula (3) or filter spectrum transform is performed by the second calculation unit according to the selected IMF band to obtain the data Z corresponding to an image plot (that is, a time frequency plot).

Lastly, the method proceeds to step 550, an image plot is correspondingly generated according to the data Z generated in step 640, the image plot is compared with cardiopathy heart-sound-plot data, and the comparison result can be used as a basis for cardiopathy discrimination. After the heart sound signal is distinguished, a physiological state information according to the image plot and/or subsequent feedback information corresponding to the physiological state information are generated. Then, the image plot is compared with a cardiopathy heart-sound-plot database by a comparison unit to obtain a comparison result signal, wherein the comparison result signal at least comprises a cardiac physiological state corresponding to an image plot. The cardiac physiological state corresponding to the detected heart-sound signal is displayed on a display unit. In step 550, the comparison result signal is delivered to the display unit by way of wireless transmission (Bluetooth transmission) or wired transmission interface (USB interface, RS232 or 1394 transmission line) to display the cardiac physiological state corresponding to the heart sound image plot. In another embodiment, the comparison result signal further comprises a subsequent treatment corresponding to the cardiac physiological state, and the step 550 further comprises displaying the subsequent treatment corresponding to the cardiac physiological state.

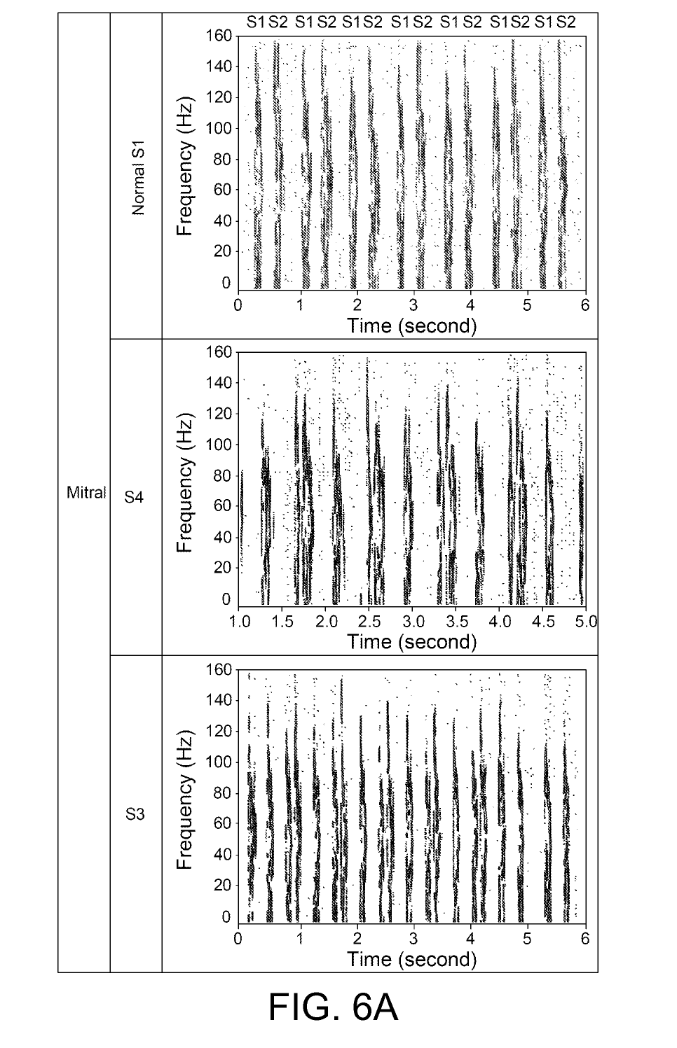

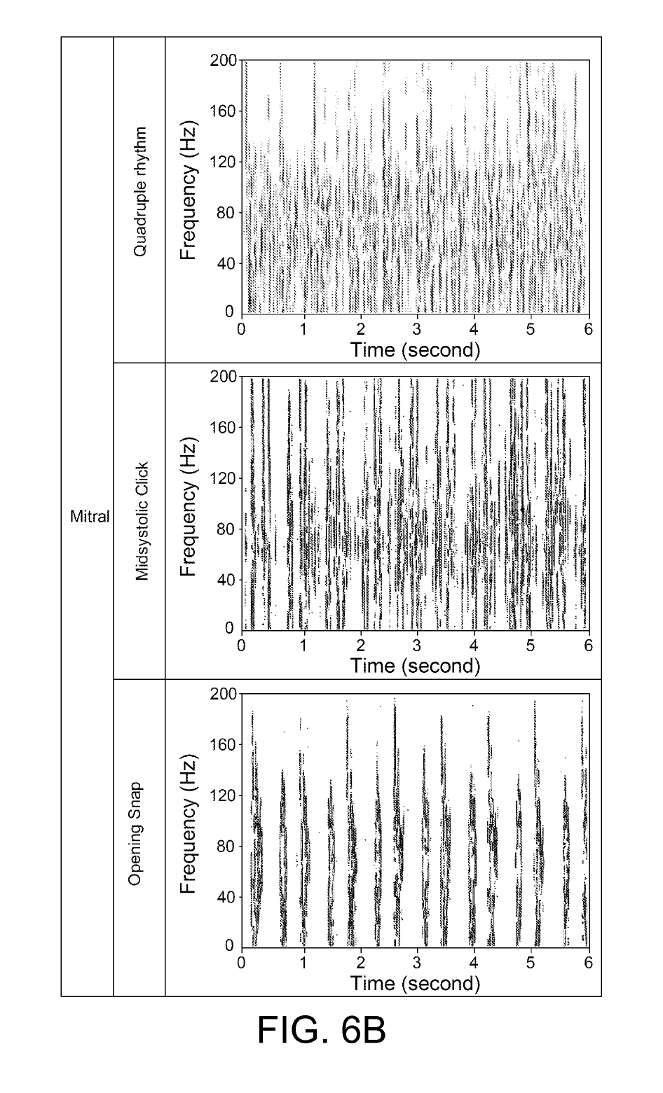

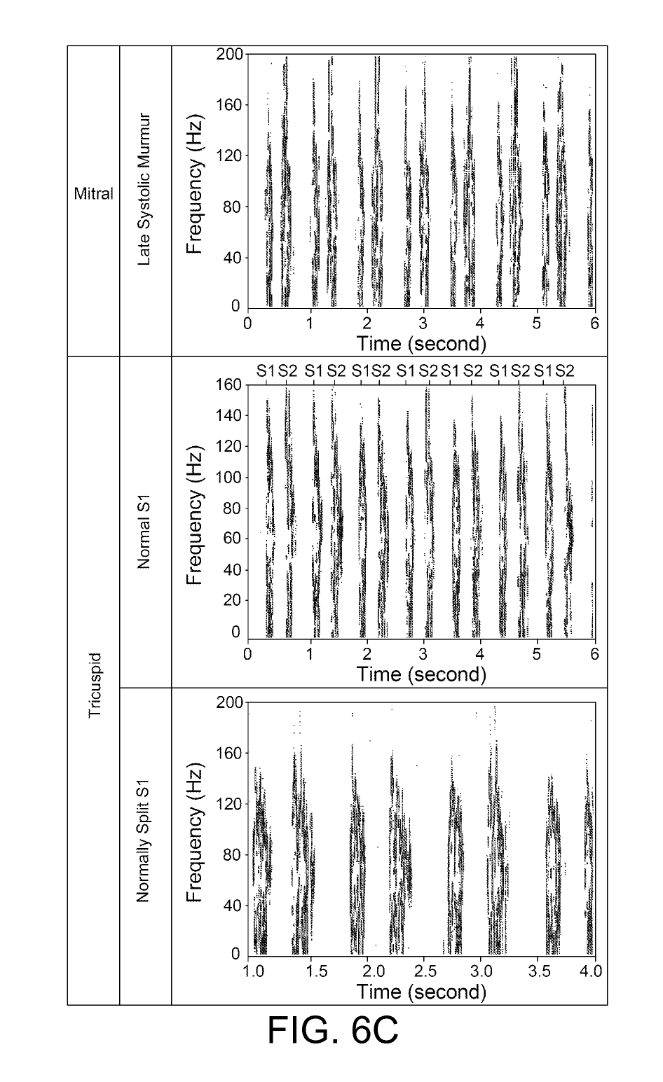

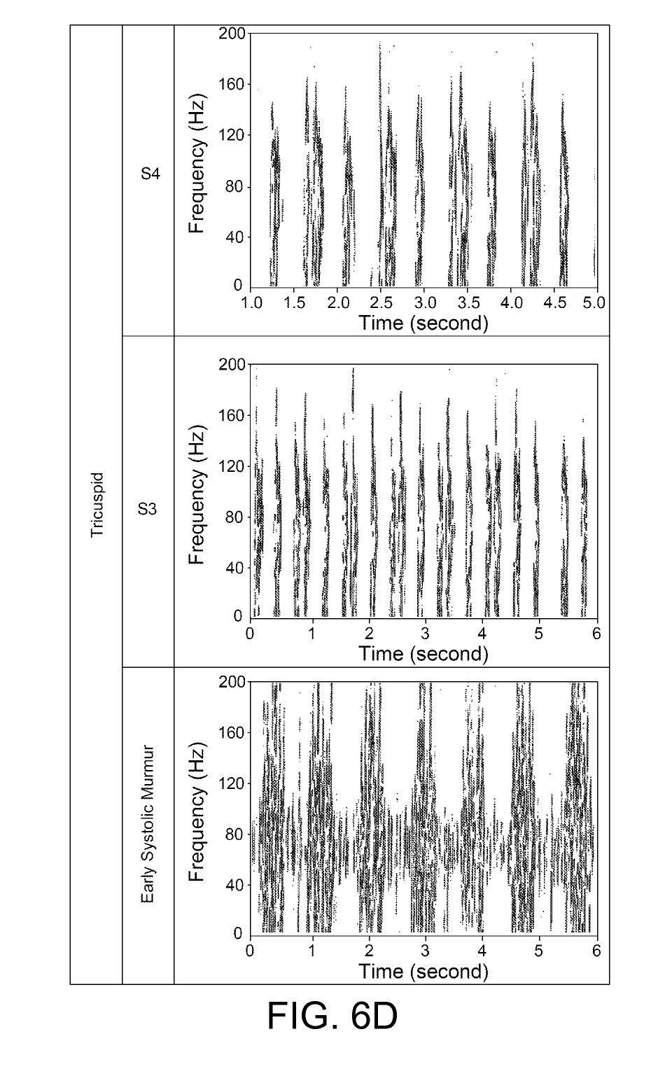

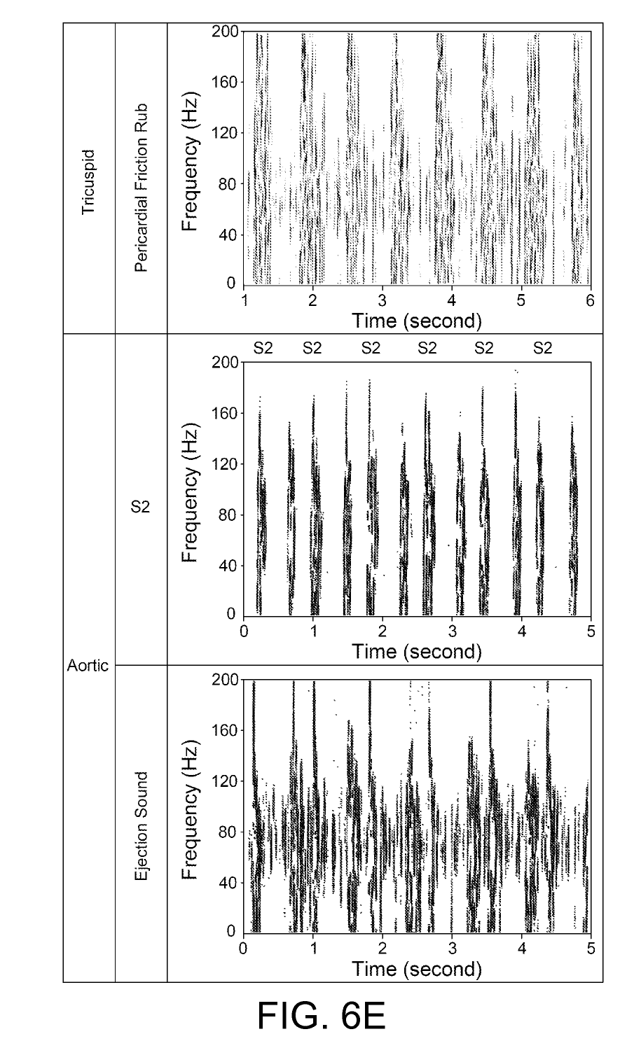

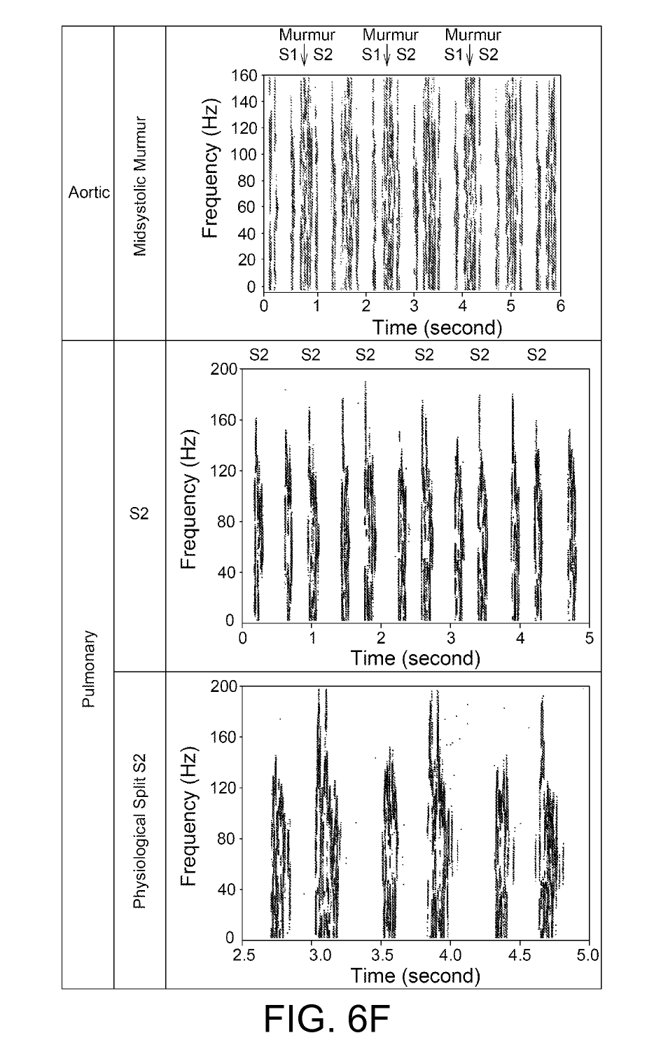

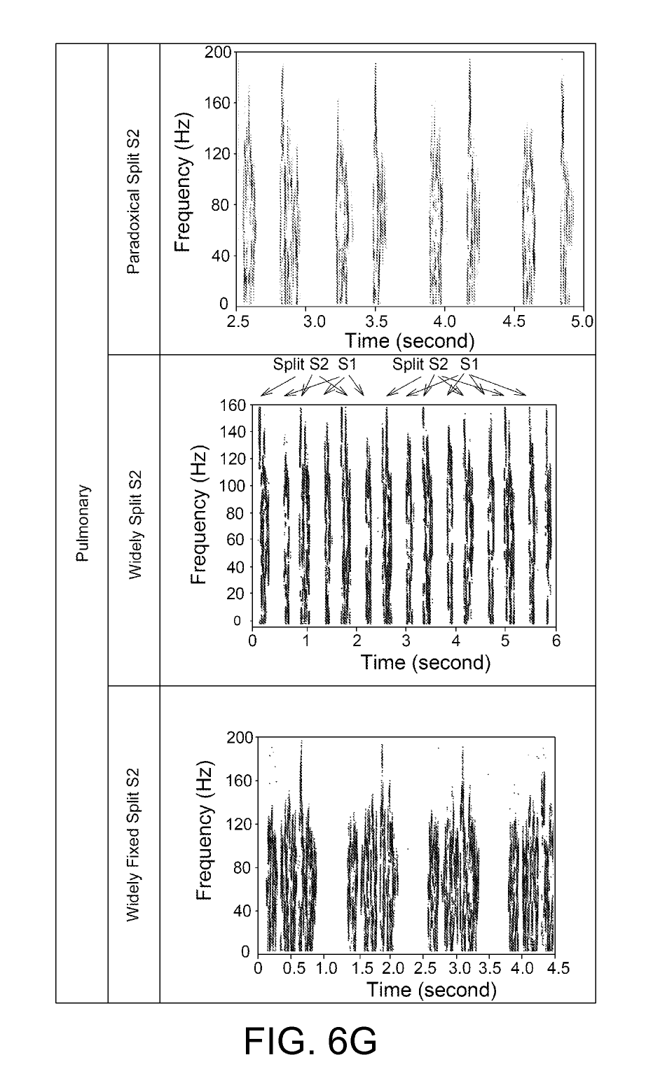

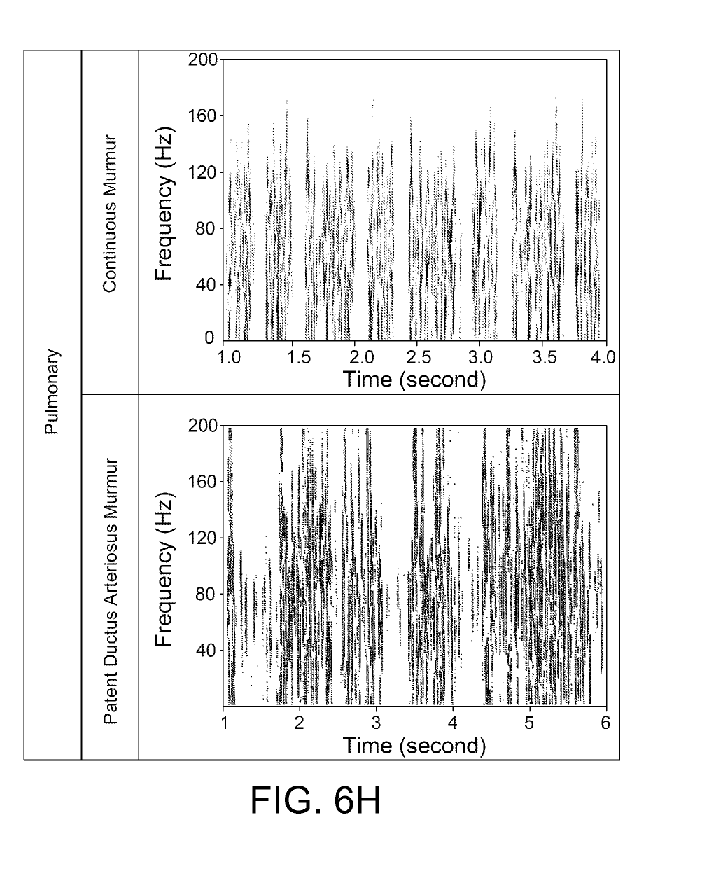

Referring to FIGS. 6A˜6H, an example of a cardiopathy heart-sound-plot database according to an exemplary embodiment of the invention are shown. As indicated in FIGS. 6A˜6H, the cardiopathy heart-sound-plot database records the corresponding time-frequency plots of the mitral-related normal first sound (S1), fourth sound (S4), third sound (S3), quadruple rhythm, midsystolic click, opening snap, and late systolic murmur, thecorresponding time-frequency plots of the tricuspid related normal S1, normally split S1, S4, S3, early systolic murmur, pericardial friction rub, and corresponding time-frequency plots of the aortic related S2, ejection sound, and midsystolic murmur, and the corresponding time-frequency plots of the pulmonary related S2, physiological split S2, paradoxical split S2, widely split S2, widely fixed split S2, continuous murmur, and patent ductus arteriosus murmur. Thus, when the detected heart-sound signal is transformed into a required time-frequency plot through calculation, the cardiopathy heart-sound-plot database can be used for comparison to promptly locate the related cardiac physiological state and perform a subsequent medical treatment of the disease.

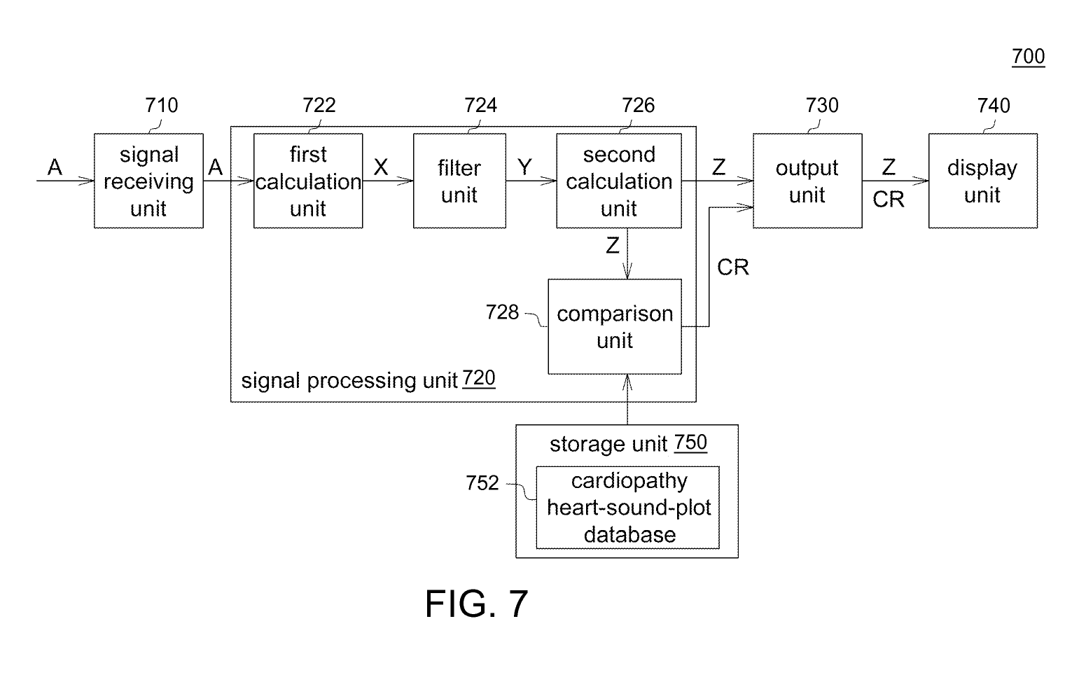

Referring to FIG. 7, a block diagram of a system for discriminating heart sound and cardiopathy according to an embodiment of the invention is shown. For example, the system for discriminating heart sound and cardiopathy is for implementing the aforementioned methods for discriminating heart sound and cardiopathy. As indicated in FIG. 7, the system 700 for discriminating heart sound and cardiopathy includes a signal receiving unit 710, a signal processing unit 720, an output unit 730, a display unit 740 and a storage unit 750. The signal receiving unit 710 is used for receiving a heart-sound signal A. The signal receiving unit 710 is realized by such as an electronic stethoscope for auscultating the patient's heart sound. Alternatively, the signal receiving unit 710 is realized by such as a signal receiver connected to an external electronic stethoscope for receiving the patient's heart-sound signal. The electronic stethoscope samples the patient's heart sound signals at different sampling frequencies such as 11025 Hz and 44100 Hz. Therefore, the heart-sound signal A comprises a plurality of heart-sound frequencies sampled within a pre-determined time (such as 5 seconds).

The signal processing unit 720, realized by such as a field programmable gate array (FPGA) processor or a central processing unit (CPU), comprises a first calculation unit 722, a filter unit 724, a second calculation unit 726 and a comparison unit 728. The first calculation unit 722 is coupled to the signal receiving unit 710 for performing a specific function calculation on the heart-sound signal A to generate a first calculation signal X, wherein the specific function calculation is based on the product of the natural log of the absolute value of the heart-sound signal A multiplied by the heart-sound signal A, such as X=cAln|A′| with c being any value or function value, A′=A if A≠0, and A′=R if A=0, R≧1, R is a real number, That is, when the sampled heart-sound signal A equals 0, the corresponding calculation signal X also equals 0.

The present embodiment of the invention suppresses the noise of the heart-sound signal, so that the to-be-detected heart-sound signal is relatively enhanced. The present embodiment of the invention is not limited to using the aforementioned specific function calculation X=cAln|A′|, and any designs using any specific functions to suppress the noise of the heart sound signal are within the spirit of the invention.

The filter unit 724, realized by such as a median filter, is coupled to the first calculation unit 722 for filtering the first calculation signal X to generate a filtering signal Y. The first calculation unit 722 suppresses the noise of the heart sound signal to generate the first calculation signal X, and the filter unit 724 further filters the first calculation signal X to effectively eliminate minor noises. The filter unit 724 can also be a Gaussian filter, a Chebyshev filter or a Bessel filter (see References [1]-[3]).

The second calculation unit 726 is coupled to the filter unit 724 for performing an HHT calculation on the filtering signal Y to generate a plurality of intrinsic mode function (IMF) bands, and generate the data Z corresponding to an image plot according to at least one of the required IMF bands. For example, the second calculation unit 126 calculates a plurality of IMF bands IMF1, IMF2, IMF3 . . . , and selects at least one IMF band conforming to the heart sound band from the IMF bands, and normally, the second digital IMF2 is selected. Then, an STFT calculation or a filter spectrum transformation is performed on the selected IMF band to obtain the data Z corresponding to the image plot. For example, the image plot is a time-frequency plot.

The output unit 730, realized by such as a wireless transmission module or a wired transmission interface, is coupled to the second calculation unit 726 for outputting the data Z corresponding to the image plot. Examples of the wireless transmission module comprise the Bluetooth transmission module, and examples of the wired transmission interface comprise the universal serial bus (USB) interface, the RS232 or the 1394 transmission line. Besides, the display unit 740, realized by such as an LCD display, is coupled to the output unit 730 for displaying the image plot (that is, the time-frequency plot) corresponding to the detected heart-sound signal. Thus, following the aforementioned specific function calculation, the time-frequency plot obtained by performing an HHT calculation and selectively performing an STFT (or filter spectrum transformation) calculation on the heart-sound signal clearly shows that the noise is reduced to a minimum or is completely eliminated, so that the discrimination in the area where the signal should occur is improved, the clinical doctor can effectively diagnose disease, the training doctor learns how to diagnose related diseases, and the heart sound signal can thus be promptly and correctly discriminated.

The signal processing unit 720 further comprises a comparison unit 728 connected to the second calculation unit 726, the storage unit 750 and the output unit 730 for comparing the image plot with the heart-sound-plot data or the cardiopathy heart-sound-plot database 752 to output a comparison result signal CR to the output unit 730. The storage unit 750 is used to store the heart-sound-plot data and the cardiopathy heart-sound-plot database 752, and the storage unit 750 is realized by such as a register or a memory.

The comparison unit 728 compares the image plot with the heart-sound-plot data to generate the comparison result CR transmitted to the display unit 724 via the output unit 730 for discriminating heart sound. In another embodiment, the comparison unit 728 compares the image plot with the cardiopathy heart-sound-plot database 752 to generate the comparison result CR transmitted to the display unit 724 via the output unit 730 for cardiopathy discrimination. The cardiopathy heart-sound-plot database 752 is such as a comparison table of the cardiopathy heart-sound plots and the cardiac physiological state constructed from the collected cardiopathy heart-sound plots and their corresponding cardiac physiological states as indicated in FIGS. 6A˜6H. In another embodiment, the cardiopathy heart-sound-plot database further records the cardiac physiological states and corresponding heart sound image plots and subsequent treatments.

The comparison result signal CR at least comprises a cardiac physiological state corresponding to the image plot. The output unit 730 outputs the comparison result signal CR to the display unit 740 for displaying the patient's cardiac physiological state. In another embodiment, the comparison result signal CR further comprises a subsequent treatment corresponding to the cardiac physiological state, and the display unit 740 further displays the subsequent treatments corresponding to various cardiac physiological states to assist the doctor to make prompt disease diagnosis and real-time subsequent medical treatments. Moreover, the system 700 for discriminating heart sound and cardiopathy of the present embodiment of the invention can be combined with the hospital electronic medical records system to form a real-time electronic medical records system.

Although the signal processing unit 720 is exemplified to output the data Z and the comparison result signal CR to the display unit 740 via the output unit 730 in the embodiment for illustration, in another embodiment, the signal processing unit 720 can also output the data Z and the comparison result signal CR directly to the display unit 740 in order to timely display the image plot and the patient's cardiac physiological state corresponding to the image plot.

According to the method and system for discriminating heart sound and cardiopathy disclosed in the aforementioned embodiment of the invention, a specific function calculation is performed on the to-be-detected heart-sound signal to effectively separate the to-be-detected heart-sound signal from the noise and generate a time-frequency plot which is easy to discriminate for the clinical doctor to make prompt diagnosis of disease or for the training doctor to learn to distinguish related diseases from the plot. The heart sound and cardiopathy discriminating system in conjunction with hardware such as electronic stethoscope and LCD display panel can be combined with the hospital electronic medical records system to form a real-time electronic medical records system that is simple, compact and portable. Furthermore, a time-frequency plot of the patient's heart sound signal can be compared with an existing cardiopathy heart-sound-plot database to assist the doctor to make prompt diagnosis of the cardiac physiological state and perform subsequent processing to achieve accurate and efficient disease diagnosis.

It will be apparent to those skilled in the art that various modifications and variations can be made to the disclosed embodiments. It is intended that the specification and examples be considered as exemplary only, with a true scope of the disclosure being indicated by the following claims and their equivalents.

### REFERENCES

[1] R. A. Haddad and A. N. Akansu, “A Class of Fast Gaussian Binomial Filters for Speech and Image Processing,” IEEE Transactions on Acoustics, Speech and Signal Processing, vol. 39, pp 723-727, March 1991
[2] Daniels, Richard W. (1974). Approximation Methods for Electronic Filter Design. New York: McGraw-Hill. ISBN 0-07-015308-6.
[3] Thomson, W. E., “Delay Networks having Maximally Flat Frequency Characteristics”, Proceedings of the Institution of Electrical Engineers, Part III, November 1949, Vol. 96, No. 44, pp. 487-490.
[4] Huang, et al. “The empirical mode decomposition and the Hilbert spectrum for nonlinear and non-stationary time series analysis.” Proc. R. Soc. Lond. A (1998) 454, 903-995.
[5] Huang, N. E., Long, S. R. and Shen, Z. 1996: The mechanism for frequency downshift in nonlinear wave evolution. Adv. Appl. Mech., 32, 59-111.
[6] Huang, N. E., Z. Shen, R. S. Long, 1999: A New View of Nonlinear Water Waves—The Hilbert Spectrum, Ann. Rev. Fluid Mech. 31, 417-457.
[7] Fourier, J. B. Joseph (1822), Théorie Analytique de la Chaleur, Paris.
[8] E. Jacobsen and R. Lyons, The sliding DFT, Signal Processing Magazine vol. 20, issue 2, pp. 74-80 (March 2003).

What is claimed is:

1. A method for discriminating heart sound, comprising:
receiving a heart-sound signal;
performing a specific function calculation on the heart-sound signal to generate a first calculation signal, wherein the specific function calculation is based on a product of a natural log of an absolute value of the heart-sound signal multiplied by the heart-sound signal;
filtering the first calculation signal to generate a filtering signal;
performing a transformation calculation on the filtering signal to generate data for an image plot; and
generating the image plot corresponding to the data generated in the step of performing the transformation calculation and comparing the image plot with data of heart-sound plots to discriminate the heart sound.

2. The method according to claim 1, wherein the specific function in the step of performing the specific function calculation is expressed as X=cAln|A′| with c being any value or function value, A′=A if A≠0, and A′=R if A=0, R≧1, R is a real number.

3. The method according to claim 1, wherein in the step of performing the transformation calculation, a Hilbert-Huang transform (HHT) calculation is performed on the filtering signal to generate a plurality of intrinsic mode function (IMF) bands and generate the data corresponding to the image plot according to at least one of the required IMF bands.

4. The method according to claim 3, wherein in the step of performing the transformation calculation, at least one IMF band conforming to a heart sound band is selected from the IMF bands, and a short time Fourier transform (STFT) calculation is performed on the selected IMF band to obtain the data corresponding to the image plot.

5. The method according to claim 3, wherein in the step of performing the transformation calculation, at least one IMF band conforming to a heart sound band is selected from the IMF bands, and filter spectrum transform is performed on the selected IMF band to obtain the data corresponding to the image plot.

6. The method according to claim 1, wherein in the step of filtering the first calculation signal, filtering is performed with a median filter.

7. The method according to claim 1, further comprising the step of:
generating a physiological state information according to the corresponding image plot and/or a subsequent feedback information corresponding to the physiological state information after the heart sound is discriminated.

8. A method for discriminating cardiopathy, comprising:
receiving a heart-sound signal;
performing a specific function calculation on the heart-sound signal to generate a first calculation signal, wherein the specific function calculation is based on a product of a natural log of an absolute value of the heart-sound signal multiplied by the heart-sound signal;
filtering the first calculation signal to generate a filtering signal;
performing a transformation calculation on the filtering signal to generate data for an image plot; and
generating the image plot corresponding to the data generated in the step of performing the transformation calculation and comparing the image plot with data of cardiopathy heart-sound plots for cardiopathy discrimination.

9. The method according to claim 8, wherein the specific function in the step of performing a specific function calculation is expressed as X=cAln|A′| with c being any value or function value, A′=A if A≠0, and A′=R if A=0, R≧1, R is a real number.

10. The method according to claim 8, wherein in the step of performing the transformation calculation, a Hilbert-Huang transform (HHT) calculation is performed on the filtering signal to generate a plurality of intrinsic mode function (IMF) bands and generate the data corresponding to the image plot according to at least one of the required IMF bands.

11. The method according to claim 10, wherein in the step of performing the transformation calculation, at least one IMF band conforming to a heart sound band is selected from the IMF bands and a short time Fourier transform (STFT) calculation is performed on the selected IMF band to obtain the data corresponding to the image plot.

12. The method according to claim 10, wherein in the step of performing the transformation calculation, at least one IMF band conforming to a heart sound band is selected from the IMF bands and a filter spectrum transformation is performed on the selected IMF band to obtain the data corresponding to the image plot.

13. The method according to claim 8, wherein in the step of filtering the first calculation signal, filtering is performed with a median filter.

14. The method according to claim 8, wherein the step of generating the image plot further comprises generating a physiological state information according to the image plot and/or subsequent feedback information corresponding to the physiological state information.

15. A system for discriminating heart sound and cardiophy, comprising:
a signal receiving unit for receiving a heart-sound signal;
a signal processing unit, comprising:
a first calculation unit coupled to the signal receiving unit for performing a specific function calculation on the heart-sound signal to generate a first calculation signal, wherein the specific function calculation is based on a product of a natural log of an absolute value of the heart-sound signal multiplied by the heart-sound signal;
a filter unit coupled to the first calculation unit for filtering the first calculation signal to generate a filtering signal; and
a second calculation unit coupled to the filter unit for performing a transformation calculation on the filtering signal to generate data for an image plot.

16. The system according to claim 15, wherein the specific function is expressed as X=cAln|A′| with c being any value or function value, A′=A if A≠0, and A′=R if A=0, R≧1, R is a real number.

17. The system according to claim 15, wherein the second calculation unit performs a Hilbert-Huang transform (HHT) calculation on the filtering signal to generate a plurality of intrinsic mode function (IMF) bands and generate the data corresponding to the image plot according to at least one of the required IMF bands.

18. The system according to claim 17, wherein the second calculation unit selects at least one IMF band conforming to a heart sound band from the IMF bands and performs a short time Fourier transform (STFT) calculation on the selected IMF band to obtain the data corresponding to the image plot.

19. The system according to claim 17, wherein the second calculation unit selects at least one IMF band conforming to the heart sound band from the IMF bands and performs filter spectrum transformation on the selected IMF band to obtain the data corresponding to the image plot.

20. The system according to claim 15, wherein the filter unit is a median filter.

21. The system according to claim 15, further comprising:
an output unit coupled to the second calculation unit for outputting the image plot or the data corresponding to the image plot.

22. The system according to claim 15, further comprising a storage unit for storing a database of heart-sound plots, wherein the signal processing unit further comprises a comparison unit for comparing the image plot with the heart-sound plots stored in the database.
